(19) United States
(12) Patent Application Publication
(10) Pub. No.: US 2026/0130737 A1
(43) Pub. Date: May 14, 2026

(54) STERILE SLEEVE BARRIER WITH INCORPORATED TUBING KIT FOR SMOKE AND FLUID EVACUATION

(71) Applicants: William J. Zinnanti, Santa Cruz, CA (US); Stacey Boscoe, Santa Cruz, CA (US)

(72) Inventors: William J. Zinnanti, Santa Cruz, CA (US); Stacey Boscoe, Santa Cruz, CA (US)

(21) Appl. No.: 19/435,940

(22) Filed: Dec. 30, 2025

Related U.S. Application Data
(62) Division of application No. 18/374,491, filed on Sep. 28, 2023.
(60) Provisional application No. 63/410,797, filed on Sep. 28, 2022.

(51) Int. Cl. A61B 50/30; A61L 2/26; A61L 103/15

(52) U.S. Cl. CPC A61B 50/30; A61L 2/26; A61B 2050/314; A61L 2103/15

(57) ABSTRACT

A packaging system comprises a sterilized medical device. An elongated flexible tubular sleeve insertably receives the sterilized medical device. A peel-open pouch sealably contains the sterilized medical device and elongated flexible tubular sleeve. In an embodiment of the invention, a tubular sleeve card is coupled to a proximal end of the flexible tubular sleeve, the tubular sleeve card surrounding at least a portion of a proximal end of the sterilized medical device.

### PRIORITY CLAIM

The present application is a divisional application of, and claims priority of the filing date of U.S. non-provisional application Ser. No. 18/374,491, filed 28 Sep. 2023, which, in turn, is a non-provisional, and claims priority under 35 U.S.C. § 119(e), of the filing date of U.S. provisional Ser. No. 63/410,797, filed 28 Sep. 2022, the complete contents and disclosure of each of which are hereby expressly incorporated by reference.

### TECHNICAL FIELD

The invention relates to surgical devices and packaging for same.

### BACKGROUND OF THE INVENTION

Most surgical procedures utilize electrosurgical devices for dissection and maintaining hemostasis. These devices produce smoke and vapors that may contain potentially infectious and/or carcinogenic byproducts. Much effort has been made to collect and control exposure to surgical smoke, however, the available devices are not readily adaptable to all electrosurgical handpieces and therefore require new equipment and greater cost. Alternatively, reusable electrosurgical handpieces are widely used mostly in the outpatient setting such as in primary care or dermatologic procedures. Many of these procedures require a sterile field such as in Mohs micrographic surgery.

Multiple designs have been produced comprising a disposable handpiece with incorporated suction, e.g., U.S. Pat. No. 5,234,428 to Kaufman and U.S. Pat. No. 8,057,470 to Lee et al. Disposable handpieces available at the time of this writing can be expensive and/or cumbersome to use. Other available devices comprise an attachable conduit or tubing system to an electrosurgical handpiece, such as U.S. Pat. No. 5,836,944 to Cosmescu; U.S. Pat. No. 5,224,944 to Elliott; and U.S. Pat. No. 6,146,353 to Platt. These devices are not adaptable to different handpieces. None of the devices listed above are available with an incorporated sterile barrier that can surround the reusable handpiece and cord.

Therefore, it would be desirable to provide an attachable suction tube or conduit kit contained within a sterile barrier sleeve that can accept a non-sterile reusable electrosurgical handpiece and provide a sterile barrier for the handpiece and cord that need to be on or near the sterile field. Additionally, the device can be provided with special electrodes that maximize the suction of smoke and debris from the surgical site.

### SUMMARY OF THE INVENTION

The current invention provides a sterile barrier sleeve with an incorporated suction conduit or tube that attaches to any electrosurgical handpiece at the distal end and to any suction source at the proximal end. The device is packaged inside a sterile peel pouch so that when opened on the proximal end, a sterile handpiece can be lowered into the package through the sterile sleeve and into the distal tip that is made of soft plastic and readily accepts and attaches to the nose or distal tip of the electrosurgical handpiece. The sterile sleeve is folded on itself so that, when the handpiece is in place, the packaging can be removed, and the proximal end of the sterile sleeve can be pulled open to expand and cover the cord and suction tubing attached to the handpiece, in a manner similar to the use of camera drapes in surgical theaters.

An embodiment of the invention comprises a packaging system comprising a sterilized medical device; an elongated flexible tubular sleeve insertably receiving the sterilized medical device; and a peel-open pouch sealably containing the sterilized medical device and elongated flexible tubular sleeve.

In an embodiment of the invention, the elongated flexible tubular sleeve has a nominal length which is substantially greater than a nominal length of the sterilized medical device. The elongated flexible tubular sleeve may be shortened from its nominal length via accordion folding.

In an embodiment of the invention, the peel-open pouch has a closed distal end and a peel-open proximal end having juxtaposed free tabs for facilitating opening of the peel-open pouch. The sterilized medical device has a distal end disposed proximate the distal end of the peel-open pouch and a proximal end disposed proximate the proximal end of the peel-open pouch. The flexible tubular sleeve includes an open distal end disposed adjacent and surrounding the distal end of the sterilized medical device such that a distal tip of the sterilized medical device projects through the open distal end of the sleeve.

In an embodiment of the invention, the peel-open pouch has first and second juxtaposed sidewalls, wherein at least one of the sidewalls is fabricated from a material that is at least one of transparent and translucent. At least a portion of the flexible tubular sleeve is fabricated from a material that is at least one of transparent and translucent.

In an embodiment of the invention, the flexible tubular sleeve comprises a stretchable elastic material such that the flexible tubular sleeve closely conforms to an outer surface of the sterilized medical device.

In an embodiment of the invention, a tubular sleeve card is coupled to a proximal end of the flexible tubular sleeve, the tubular sleeve card surrounding at least a portion of a proximal end of the sterilized medical device. The tubular sleeve card further comprises a tab to facilitate grasping by a user. In an embodiment of the invention, the tubular sleeve card comprises a blank of material formed into a tubular structure. The blank of material may comprise at least one of paper, paperboard, card stock, corrugated paperboard, plastic.

A method of providing a sterile sleeve barrier with incorporated tubing, comprising the steps of:
providing at least one previously-sterilized piece of surgical equipment, the at least one previously-sterilized piece of surgical equipment including tubing;
positioning the at least one previously-sterilized piece of surgical equipment into a sleeve, wherein the sleeve is axially-shortened, such that when the sleeve is fully extended, it has an overall length that is substantially greater than a total length of the at least one piece of surgical equipment;
positioning the at least one previously-sterilized piece of surgical equipment, within the sleeve, into an external pouch; and
sealing the external pouch.

The external pouch may include first and second juxtaposed sheets of material, the first and second juxtaposed sheets of material being releasably and sterilizably couplable to one another.

In embodiments of the invention, the method further comprises one or more of the following steps:
fabricating one of the first and second sheets of the pouch from transparent or translucent material;
fabricating the other of the first and second sheets of the pouch from an opaque material;
fabricating the first and second sheets of the pouch from a sterilizable material;
joining the first and second sheets of the pouch together via one of an adhesive, a cohesive, a heat-sealing mechanism which can withstand sterilization without separation or compromise of the seal formed therebetween, such that the sterilized state may be maintained for a predetermined minimum period of time, but which first and second sheets of the pouch may be separated by peeling after sterilization;
inserting a sleeve card into an end of the sleeve;
coupling the sleeve card to a distal end of the sleeve, wherein the coupling is accomplished by one or more of providing the sleeve card with a width greater than a nominal width of the sleeve, affixing the sleeve card to the sleeve with adhesive tape, applying an adhesive or cohesive material to one or both of the sleeve card and the sleeve, providing barb-shaped cutouts on the sleeve card.

An embodiment of the invention further comprises a method of use of a sterile sleeve barrier, comprising the steps of:
providing a package containing at least one piece of surgical equipment, the package including the at least one piece of surgical equipment surrounded by an axially-shortened, extensible barrier sleeve, the at least one piece of surgical equipment and sleeve being enclosed within a sealed pouch, the package being one of sterilizable or previously sterilized, the sealed pouch comprising first and second juxtaposed sheets of material being peelably affixed to one another;
opening the package;
removing the at least one piece of surgical equipment and barrier sleeve from the package;
coupling the at least one piece of surgical equipment to a second piece of surgical equipment;
extending the barrier sleeve to at least partially cover the second piece of surgical equipment.

In embodiments of the invention the method of use of a sleeve barrier further comprises at least one of:
inserting a sleeve card into the sleeve and affixing the sleeve card to the sleeve, prior to enclosing the at least one piece of surgical equipment and the barrier sleeve in the pouch;
grasping the sleeve card, after opening the pouch, to extend the barrier sleeve.

An embodiment of the invention comprises a sterilizable packaging system, comprising a medical device; an elongated flexible tubular sleeve insertably receiving the medical device; and a peel-open pouch sealably containing the medical device and elongated flexible tubular sleeve; and a tubular sleeve card coupled to a proximal end of the flexible tubular sleeve, the tubular sleeve card surrounding at least a portion of a proximal end of the medical device.

In an embodiment of the sterilizable packaging system, the tubular sleeve card further comprises a tab to facilitate grasping by a user.

In an embodiment of the sterilizable packaging system, the tubular sleeve card comprises a blank of material formed into a tubular structure.

In an embodiment of the sterilizable packaging system, the blank of material comprises at least one of paper, paperboard, card stock, corrugated paperboard, a plastic material.

In an embodiment of the sterilizable packaging system, the elongated flexible tubular sleeve has a nominal length which is substantially greater than a nominal length of the medical device.

In an embodiment of the sterilizable packaging system, the elongated flexible tubular sleeve is shortened from its nominal length via accordion folding.

In an embodiment of the sterilizable packaging system, the peel-open pouch having a closed distal end and a peel-open proximal end having juxtaposed free tabs for facilitating opening of the peel-open pouch; and the medical device having a distal end disposed proximate the distal end of the peel-open pouch and a proximal end disposed proximate the proximal end of the peel-open pouch; and the flexible tubular sleeve includes an open distal end disposed adjacent and surrounding the distal end of the medical device such that a distal tip of the medical device projects through the open distal end of the sleeve.

In an embodiment of the sterilizable packaging system, the peel-open pouch having first and second juxtaposed sidewalls, wherein at least one of the sidewalls is fabricated from a material that is at least one of transparent and translucent; and at least a portion of the flexible tubular sleeve is fabricated from a material that is at least one of transparent and translucent.

In an embodiment of the sterilizable packaging system, the flexible tubular sleeve comprises a stretchable elastic material such that the flexible tubular sleeve closely conforms to an outer surface of the medical device.

In an embodiment of the invention, a method of providing a sterile sleeve barrier with incorporated tubing comprises the steps of:
providing at least one piece of surgical equipment, the at least one piece of surgical equipment including tubing;
positioning the at least one piece of surgical equipment into a sleeve, wherein the sleeve is axially-shortened, such that when the sleeve is fully extended, it has an overall length that is substantially greater than a total length of the at least one piece of surgical equipment;
inserting a sleeve card into an end of the sleeve;
coupling the sleeve card to a distal end of the sleeve, wherein the coupling is accomplished by one or more of providing the sleeve card with a width greater than a nominal width of the sleeve, affixing the sleeve card to the sleeve with adhesive tape, applying an adhesive or cohesive material to one or both of the sleeve card and the sleeve, providing barb-shaped cutouts on the sleeve card;
positioning the at least one piece of surgical equipment, within the sleeve, into an external pouch; and
sealing the external pouch.

In an embodiment of the method, wherein the external pouch includes first and second juxtaposed sheets of material, the first and second juxtaposed sheets of material being releasably and sterilizably couplable to one another.

In an embodiment, the method further comprises the steps of:
joining the first and second sheets of the pouch together via one of an adhesive, a cohesive, a heat-sealing mechanism which can withstand sterilization without separation or compromise of the seal formed therebetween, such that the sterilized state may be maintained for a predetermined minimum period of time, but which first and second sheets of the pouch may be separated by peeling after sterilization.

In an embodiment of the invention, the method further comprises the steps of:
fabricating one of the first and second sheets of the pouch from transparent or translucent material; and
fabricating the other of the first and second sheets of the pouch from an opaque material.

An embodiment of the invention further comprises a method of use of a sterile sleeve barrier, comprising the steps of:
providing a package containing at least one piece of surgical equipment, the package including the at least one piece of surgical equipment surrounded by an axially-shortened, extensible barrier sleeve, the at least one piece of surgical equipment and sleeve being enclosed within a sealed pouch, the package being one of sterilizable or previously sterilized, the sealed pouch comprising first and second juxtaposed sheets of material being peelably affixed to one another;
opening the package;
removing the at least one piece of surgical equipment and barrier sleeve from the package;
coupling the at least one piece of surgical equipment to a second piece of surgical equipment;
extending the barrier sleeve to at least partially cover the second piece of surgical equipment.

In an embodiment, the method of use of a sleeve barrier further comprises at least one of:
inserting a sleeve card into the sleeve and affixing the sleeve card to the sleeve, prior to enclosing the at least one piece of surgical equipment and the barrier sleeve in the pouch;
grasping the sleeve card, after opening the pouch, to extend the barrier sleeve.

The aforementioned and other features and advantages of the invention will become further apparent from the following detailed description of the presently preferred embodiments, read in conjunction with the accompanying drawings, which are not to scale. The detailed description and drawings are merely illustrative of the invention, rather than limiting, the scope of the invention being defined by the appended claims and equivalents thereof.

### BRIEF DESCRIPTION OF THE DRAWINGS

FIG. 1 is a plan view of an embodiment of the invention, illustrating a medical device contained within a peel-open pouch.

FIG. 2 is a view of the embodiment of FIG. 1, showing the pouch partially opened.

FIG. 3 is a view of the embodiment of FIG. 1, showing a device, such as an electrosurgical pencil or handpiece, being attached to the medical device contained within the pouch.

FIG. 4 is a view of the embodiment of FIG. 3, showing a suction tube being attached to the medical device within the pouch.

FIG. 5 is a view of the embodiment of FIG. 1, showing the pouch removed and the sleeve being deployed.

FIG. 6 is a view of the embodiment of FIG. 1, wherein an electrode tip is being attached to the medical device.

FIG. 7 is a plan view of an alternative embodiment of the invention, illustrating a sleeve card coupled to the flexible tubular sleeve.

FIG. 8 is an enlarged view of the embodiment of FIG. 7, showing the pouch partially opened.

FIG. 9 is an enlarged view of the flexible tubular sleeve and sleeve card, according to the embodiment of FIG. 7.

FIG. 10 is an end view of the sleeve card according to the embodiment of FIG. 7.

FIG. 11 is a plan view of a blank for forming a sleeve card according to the embodiment of FIG. 7.

### DETAILED DESCRIPTION OF THE DRAWINGS

While this invention is susceptible of embodiment in many different forms, there are shown in the drawings and described in detail herein, specific embodiments, with the understanding that the present disclosure is to be considered as an exemplification of the principles of the invention, and is not intended to limit the invention to the embodiment(s) illustrated.

The invention and accompanying drawings will now be discussed in reference to the numerals provided therein so as to enable one skilled in the art to practice the present invention. The drawings and descriptions are exemplary of various aspects of the invention and are not intended to narrow the scope of the appended claims. Unless specifically noted, it is intended that the words and phrases in the specification and the claims be given their plain, ordinary and accustomed meaning to those of ordinary skill in the applicable arts. It is noted that the inventors can be their own lexicographers. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors'intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f) or pre-AIA 35 U.S.C. § 112˜6. Thus, the use of the words “function,” “means” or “step” in the Detailed Description of the Invention or claims is not intended to somehow indicate a desire to invoke the special provisions of 35 U.S.C. § 112(f) or pre-AIA 35 U.S.C. § 112˜6 to define the invention. To the contrary, if the provisions of 35 U.S.C. § 112(f) or pre-AIA 35 U.S.C. § 112˜6 are sought to be invoked to define the inventions, the claims will specifically and expressly state the exact phrases “means for” or “step for” and the specific function (e.g., “means for roasting”), without also reciting in such phrases any structure, material or act in support of the function. Thus, even when the claims recite a “means for . . . ” or “step for . . . ” if the claims also recite any structure, material or acts in support of that means or step, or that perform the recited function, then it is the clear intention of the inventor not to invoke the provisions of 35 U.S.C. §112(f) or pre-AIA 35 U.S.C. § 112˜6. Moreover, even if the provisions of 35 U.S.C. § 112(f) or pre-AIA 35 U.S.C. § 112˜6 are invoked to define the claimed inventions, it is intended that the inventions not be limited only to the specific structure, material or acts that are described in the illustrated embodiments, but in addition, include any and all structures, materials or acts that perform the claimed function as described in alternative embodiments or forms of the invention, or that are well known present or later-developed, equivalent structures, material or acts for performing the claimed function.

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and apparatus are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, apparatus and technologies to which the disclosed inventions may be applied. Thus, the full scope of the inventions is not limited to the examples that are described below.

Various aspects of the present invention may be described in terms of functional block components and various processing steps. Such functional blocks may be realized by any number of hardware or software components configured to perform the specified functions and achieve the various results.

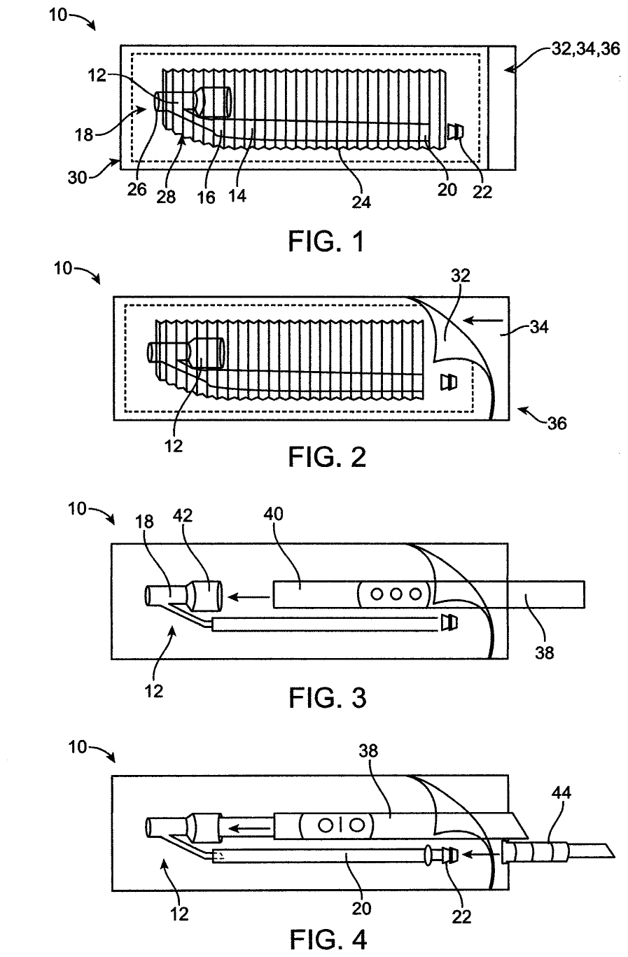

FIG. 1 illustrates an embodiment of the invention comprising a package 10 containing a sterile device 12 with a suction conduit or tube 14 having a distal end 16 attached to a soft plastic “Y” connector 18 and a proximal end 20 having a hard plastic barbed connector 22 to facilitate attachment to standard suction tubing (not shown). Device 12 is contained inside a sleeve 24 with an extreme distal tip 26 of “Y” connector 18 projecting through a tapered end 28 of sleeve 24. A proximal end of sleeve 24 is folded back on itself to facilitate introduction of a handpiece into sleeve 24, as discussed in further detail hereinafter. Device 12 is packaged inside a sterile peel-open pouch 30, having flap ends 32, 34, to facilitate opening of pouch 30 at its proximal end 36.

FIG. 2 illustrates package 10 with flap ends 32, 34 separated to begin the peeling process to open pouch 30 to enable the insertion of a tool, such as electrosurgical pencil 38 (FIG. 3). In an embodiment of the invention, pouch 30 may be fabricated from any suitable material or combination of materials. For example, in an embodiment, pouch 30 is fabricated from two sheets of polymeric material, one of which is preferably transparent or translucent, and the other of which is opaque. In an embodiment of the invention, one or both sheets is coated with a cohesive or releasable adhesive material, such that the two sheets can be sealingly and sterilizably coupled together, and then later peeled apart.

FIG. 3 illustrates package 10, with sleeve 24 omitted to facilitate illustration. Electrosurgical pencil 38 is inserted through opened proximal end 36 of pouch 30 and coupled at its distal end 40 to proximal end 42 of “Y” connector 18.

FIG. 4 illustrates package 10, with sleeve 24 omitted to facilitate illustration. Electrosurgical pencil 38 is coupled to “Y” connector 18, and further illustrating attachment of a standard suction tubing 44 to barbed connector 22 of proximal end 20 of tube 14.

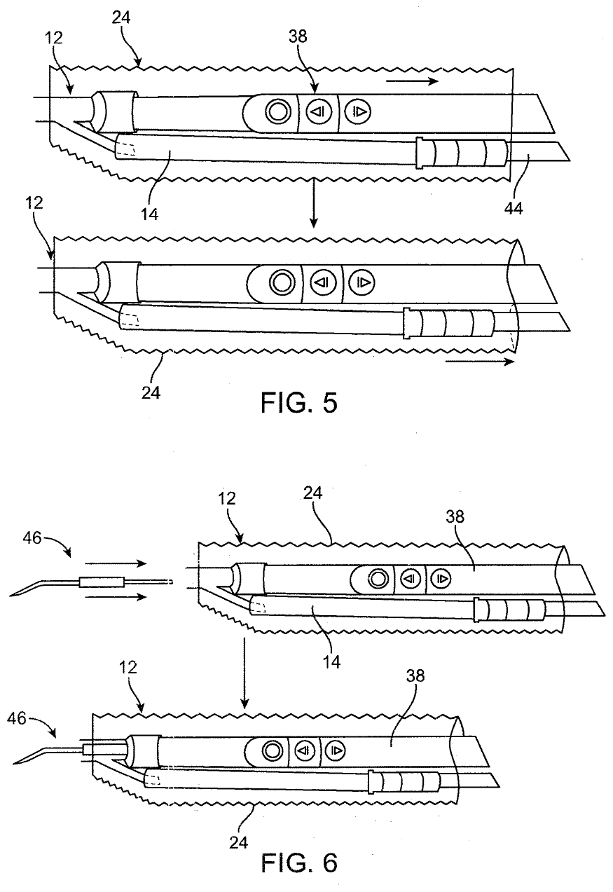

FIG. 5 illustrates device 12 removed from pouch 30. Sleeve 24 (shown only in the upper portion of FIG. 5 but understood to be present in the lower portion of FIG. 5), which may be a straight simple length of flattened tube, or which may be longitudinally-collapsed or accordion-folded while inside pouch 30, is pulled in the proximal direction, indicated by the rightward-pointing arrow in FIG. 5, to cover a cord (not shown) extending in the proximal direction from electrosurgical pencil 38 as well as the suction tubing.

FIG. 6 illustrates device 12, coupled to electrosurgical pencil 18, having an electrode tip 48 or other end effector coupled thereto, prior to the start of a surgical procedure. Sleeve 24 is omitted from FIG. 6 to facilitate illustration, but is understood to be present during use of device 12/pencil 18.

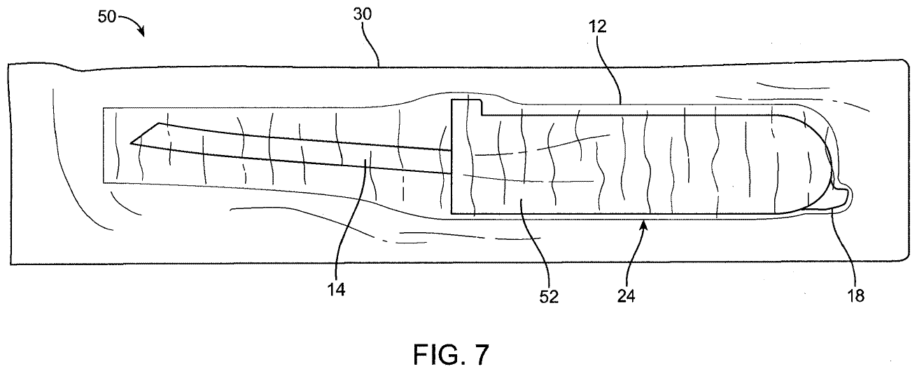

FIGS. 7-11 illustrate an alternative embodiment of the invention. Except as otherwise described hereinafter, the embodiment of FIGS. 7-11 includes or would include elements identical to those found in the embodiment of FIGS. 1-6, and accordingly, like reference numerals to those from the embodiment of FIGS. 1-6 will be employed in the description of the embodiment of FIGS. 7-11 where appropriate.

It may be observed that in surgical theaters or other medical operation situations, doctors, nurses and other personnel typically will have donned gloves, which may make grasping of thin flexible members, such as sleeve 24, difficult. Accordingly, it would be desirable to provide a mechanism to facilitate the extension of sleeve 24, without requiring the doctor, nurse, etc. to directly make contact with sleeve 24.

Accordingly, and referring specifically to FIG. 7, package 50 includes a device 12, which further includes a “Y” connector 18 coupled to a tube 14, and enclosed by a sleeve 30. Sleeve 30, in turn, is coupled to sleeve card 52.

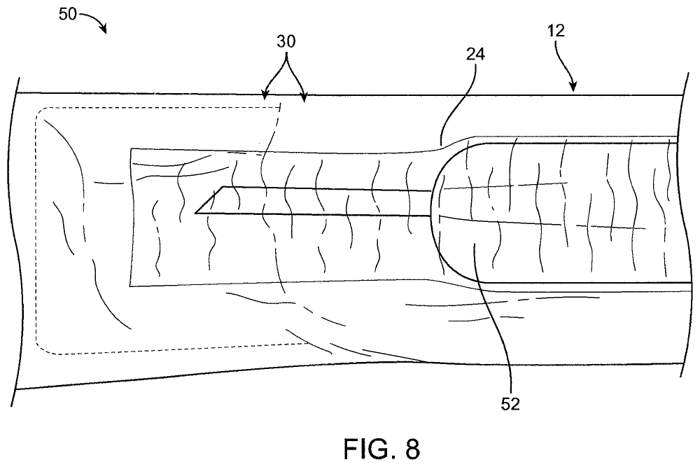

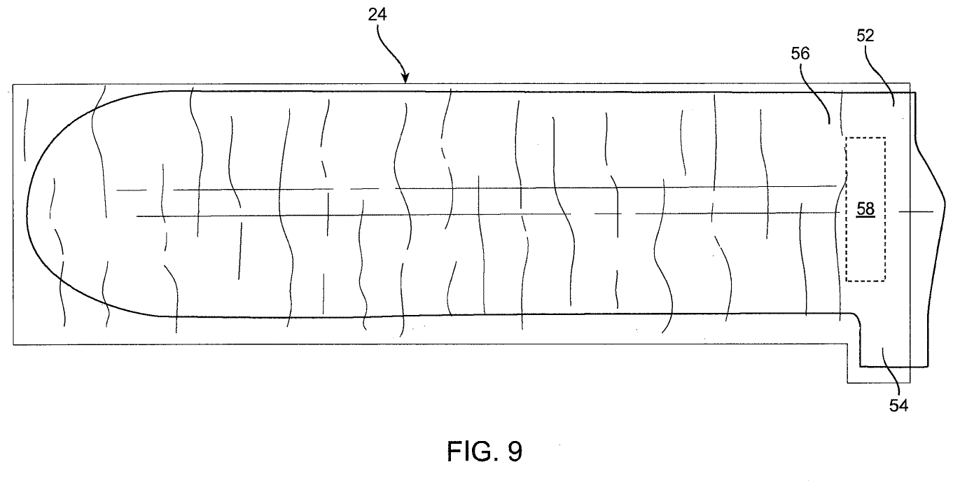

FIG. 8 is an enlarged view of package 50, with pouch 30 partially peeled open, revealing device 12, and sleeve 24 shortened via accordion-folding. FIG. 9 is an enlarged view of a proximal end of sleeve 24 and sleeve card 52, separate from device 12. Sleeve card 52 is provided with tab 54 to facilitate grasping of card 52. In an embodiment of the invention, a proximal end 56 of sleeve 24 is affixed to sleeve card 52 by any suitable means, such as adhesive tape 58. Alternative means may include placing an adhesive or cohesive material on an external surface of sleeve card 52, or providing barb-like cutouts on card 52 to engage with sleeve 24, e.g., possibly through apertures placed in proximal end 56 of sleeve 24.

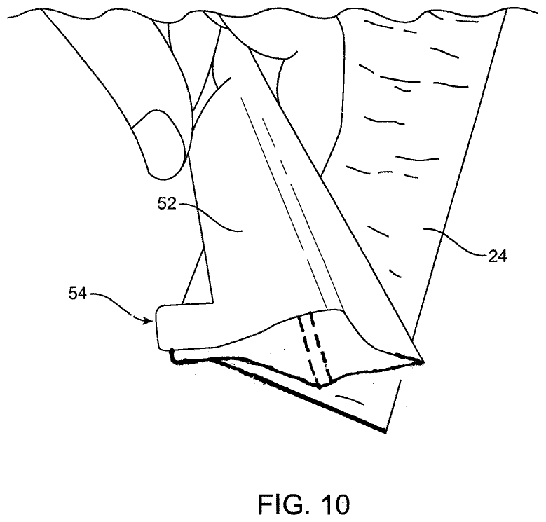

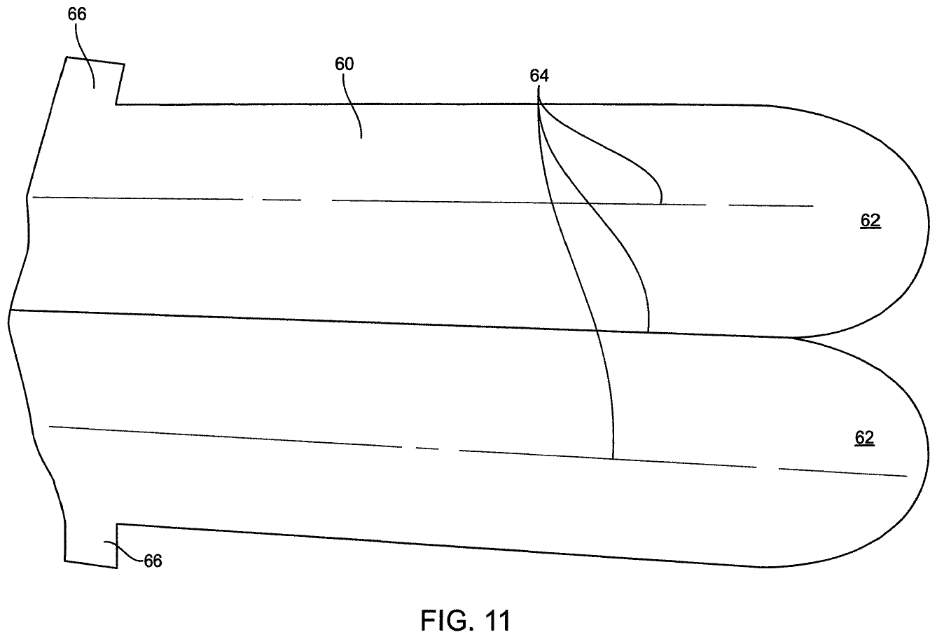

FIGS. 10-11 illustrate an exemplary configuration of sleeve card 52. Card 52 is, in an embodiment of the invention, formed from a flat blank 60 (FIG. 11) fabricated from at least one of relatively stiff card stock, paperboard material, corrugated paperboard material, plastic material and/or other sterilizable material. Blank 60 includes rounded distal portions 62, lines of weakness 64, and tab portions 66. Lines of weakness 64 may be creases, score lines, perforations, etc. Upon articulation, blank 60 forms a tubular structure having a generally diamond-shaped cross-section, as shown in FIG. 10. Rounded portions 62 facilitate the placement of sleeve 24 onto and around sleeve card. Tab portions 66 align to form tab 54.

The present invention further comprises a method of providing a sterile sleeve barrier with incorporated tubing kit. Referring to FIGS. 1-11, the method comprises:
providing at least one previously-sterilized piece of surgical equipment, such as sterile device 12, conduit 14 and/or “Y” connector 18;
positioning the at least one previously-sterilized piece of surgical equipment into a sleeve 24, wherein the sleeve 24 is axially-shortened, such that when the sleeve 24 is fully extended, it has an overall length that is substantially greater than a total length of the sterile device 12, conduit 14 and/or “Y” connector 18, but when axially-shortened, sleeve 24 has a length which is slightly greater than that of sterile device 12, conduit 14 and/or “Y” connector;
positioning the at least one previously-sterilized piece of surgical equipment, within sleeve 24, into an external pouch, e.g., pouch 30, wherein the external pouch includes first and second juxtaposed sheets of material, the first and second juxtaposed sheets of material being releasably and sterilizably couplable to one another; and
sealing the external pouch.

In an embodiment of the invention, the aforementioned method may further comprise one or more of the following:
one of the first and second sheets of the pouch being fabricated from transparent or translucent material;
the other of the first and second sheets of the pouch being fabricated from an opaque material;
the first and second sheets of the pouch being fabricated from a sterilizable material;
the first and second sheets of the pouch being joined together via an adhesive or by a heat-sealing means which can withstand sterilization without separation or compromise of the seal formed therebetween, such that the sterilized state may be maintained for a predetermined minimum period of time, but which first and second sheets of the pouch may be separated by peeling after sterilization;
inserting a sleeve card into an end of the sleeve;
coupling the sleeve card to a distal end of the sleeve, wherein the coupling is accomplished by one or more of providing the sleeve card with a width greater than a nominal width of the sleeve, affixing the sleeve card to the sleeve with adhesive tape, applying an adhesive or cohesive material to one or both of the sleeve card and the sleeve, providing barb-shaped cutouts on the sleeve card.

An embodiment of the invention further comprises a method of use of a sterile sleeve barrier, comprising the steps of:
providing a package containing at least one piece of surgical equipment, the package including the at least one piece of surgical equipment surrounded by an axially-shortened, extensible barrier sleeve, the at least one piece of surgical equipment and sleeve being enclosed within a sealed pouch, the package being one of sterilizable or previously sterilized, the sealed pouch comprising first and second juxtaposed sheets of material being peelably affixed to one another;
opening the package;
removing the at least one piece of surgical equipment and barrier sleeve from the package;
coupling the at least one piece of surgical equipment to a second piece of surgical equipment;
extending the barrier sleeve to at least partially cover the second piece of surgical equipment.

The method of use of a sleeve barrier may further comprise one or more of the steps of:
inserting a sleeve card into the sleeve and affixing the sleeve card to the sleeve, prior to enclosing the at least one piece of surgical equipment and the barrier sleeve in the pouch;
grasping the sleeve card, after opening the pouch, to extend the barrier sleeve.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

Although the invention has been described with reference to the above examples, it will be understood that many modifications and variations are contemplated within the true spirit and scope of the embodiments of the invention as disclosed herein. Many modifications and other embodiments of the invention set forth herein will come to mind to one skilled in the art to which the invention pertains having the benefit of the teachings presented in the foregoing descriptions and the associated drawings. Therefore, it is to be understood that the invention shall not be limited to the specific embodiments disclosed and that modifications and other embodiments are intended and contemplated to be included within the scope of the appended claims. Although specific terms are employed herein, they are used in a generic and descriptive sense only and not for purposes of limitation.

What is claimed is:

1. A sterilizable packaging system comprising:
a medical device;
an elongated flexible tubular sleeve insertably receiving the medical device; and
a peel-open pouch sealably containing the medical device and elongated flexible tubular sleeve; and
a tubular sleeve card coupled to a proximal end of the flexible tubular sleeve, the tubular sleeve card surrounding at least a portion of a proximal end of the medical device.

2. The packaging system according to claim 1, wherein the tubular sleeve card further comprises a tab to facilitate grasping by a user.

3. The packaging system according to claim 1, wherein the tubular sleeve card comprises a blank of material formed into a tubular structure.

4. The packaging system according to claim 3, wherein the blank of material comprises at least one of paper, paperboard, card stock, corrugated paperboard, a plastic material.

5. The packaging system according to claim 1, wherein the elongated flexible tubular sleeve has a nominal length which is substantially greater than a nominal length of the medical device.

6. The packaging system according to claim 5, wherein the elongated flexible tubular sleeve is shortened from its nominal length via accordion folding.

7. The packaging system according to claim 1, further comprising:
the peel-open pouch having a closed distal end and a peel-open proximal end having juxtaposed free tabs for facilitating opening of the peel-open pouch;
the medical device having a distal end disposed proximate the distal end of the peel-open pouch and a proximal end disposed proximate the proximal end of the peel-open pouch; and
the flexible tubular sleeve includes an open distal end disposed adjacent and surrounding the distal end of the medical device such that a distal tip of the medical device projects through the open distal end of the sleeve.

8. The packaging system according to claim 1, further comprising:
the peel-open pouch having first and second juxtaposed sidewalls, wherein at least one of the sidewalls is fabricated from a material that is at least one of transparent and translucent;
at least a portion of the flexible tubular sleeve is fabricated from a material that is at least one of transparent and translucent.

9. The packaging system according to claim 1, further comprising:
the flexible tubular sleeve comprises a stretchable elastic material such that the flexible tubular sleeve closely conforms to an outer surface of the medical device.

10. A method of providing a sterile sleeve barrier with incorporated tubing, comprising the steps of:
providing at least one piece of surgical equipment, the at least one piece of surgical equipment including tubing;
positioning the at least one piece of surgical equipment into a sleeve, wherein the sleeve is axially-shortened, such that when the sleeve is fully extended, it has an overall length that is substantially greater than a total length of the at least one piece of surgical equipment;
inserting a sleeve card into an end of the sleeve;
coupling the sleeve card to a distal end of the sleeve, wherein the coupling is accomplished by one or more of providing the sleeve card with a width greater than a nominal width of the sleeve, affixing the sleeve card to the sleeve with adhesive tape, applying an adhesive or cohesive material to one or both of the sleeve card and the sleeve, providing barb-shaped cutouts on the sleeve card;
positioning the at least one piece of surgical equipment, within the sleeve, into an external pouch; and
sealing the external pouch.

11. The method according to claim 10, wherein the external pouch includes first and second juxtaposed sheets of material, the first and second juxtaposed sheets of material being releasably and sterilizably couplable to one another.

12. The method according to claim 11, further comprising the steps of:
joining the first and second sheets of the pouch together via one of an adhesive, a cohesive, a heat-sealing mechanism which can withstand sterilization without separation or compromise of the seal formed therebetween, such that the sterilized state may be maintained for a predetermined minimum period of time, but which first and second sheets of the pouch may be separated by peeling after sterilization.

13. The method according to claim 11, further comprising the steps of:
fabricating one of the first and second sheets of the pouch from transparent or translucent material; and
fabricating the other of the first and second sheets of the pouch from an opaque material.

14. A method of use of a sterile sleeve barrier, comprising the steps of:
providing a package containing at least one piece of surgical equipment, the package including the at least one piece of surgical equipment surrounded by an axially-shortened, extensible barrier sleeve, the at least one piece of surgical equipment and sleeve being enclosed within a sealed pouch, the package being one of sterilizable or previously sterilized, the sealed pouch comprising first and second juxtaposed sheets of material being peelably affixed to one another;
opening the package;
removing the at least one piece of surgical equipment and barrier sleeve from the package;
coupling the at least one piece of surgical equipment to a second piece of surgical equipment;
extending the barrier sleeve to at least partially cover the second piece of surgical equipment.

15. The method of use of a sleeve barrier according to claim 14, further comprising at least one of:
inserting a sleeve card into the sleeve and affixing the sleeve card to the sleeve, prior to enclosing the at least one piece of surgical equipment and the barrier sleeve in the pouch;
grasping the sleeve card, after opening the pouch, to extend the barrier sleeve.
